(19) United States
(12) Patent Application Publication
(10) Pub. No.: US 2020/0242877 A1
(43) Pub. Date: Jul. 30, 2020

(54) AUTOMATED SLICED FOOD VENDING KIOSK

(71) Applicant: Walmart Apollo, LLC, Bentonville, AR (US)

(72) Inventors: Jeremy L. Velten, Bella Vista, AR (US); Jason Bellar, Bella Vista, AR (US); Brandon Joel Easterling, Bella Vista, AR (US)

(21) Appl. No.: 16/722,368

(22) Filed: Dec. 20, 2019

Related U.S. Application Data
(60) Provisional application No. 62/799,031, filed on Jan. 30, 2019.

(51) Int. Cl. G07F 11/66; G07F 9/10; G07F 9/02; G07F 11/00; G07F 11/72; G07F 17/00; A61L 2/10

(52) U.S. Cl. CPC G07F 11/66; G07F 9/105; G07F 9/026; A61L 2/10; G07F 11/72; G07F 17/0064; G07F 11/002

(57) ABSTRACT

An automated sliced food vending kiosk provides a sanitary, sealed environment for storing, slicing, packaging, and dispensing sliced food items, such as deli meats and cheeses. Orders may be customized through a user interface, and be scheduled for preparation to be completed coincidental with an expected approximate retrieval time. In some examples, wireless capability detects customer arrival, which is used as a trigger to begin preparing an earlier-submitted order. Food safety may be improved by correlating food item chubs with customer profiles, to provide alert recipient information, if needed. The kiosk may be tied in with retail inventory management for automated reordering of food items and reconciling slicing operations with sales and inventory levels.

### BACKGROUND

Retail delicatessens (delis) slice blocks of meats and cheeses (called chubs) based on customer orders. Unfortunately, this common procedure has some disadvantages. It is labor intensive, requiring the deli counter to be staffed whenever a customer wishes to order sliced food items. Because the ordering occurs in person, the customer typically does not disclose identity and contact information. If shortly after the customer receives the sliced food items and leaves, there is an alert regarding a food safety issue, it may be prohibitively difficult to locate and alert the customer immediately. Additionally, because the chubs are sliced by humans, who may not record the precise amount used for each order, reconciling inventory consumption with the number of processed orders will be challenging. Deli workers may be overly generous with portions, thereby using inventory at a faster rate than reflected by the number of orders processed by the register. Conversely, deli workers may accidentally fail to provide customers with the entirety of the food purchased.

### SUMMARY

Systems and methods are disclosed that enable an automated sliced food vending kiosk to provide a sanitary, sealed environment for storing, slicing, packaging, and dispensing sliced food items, such as deli meats and cheeses. Orders may be customized through a user interface, and be scheduled for preparation to be completed coincidental with an expected approximate retrieval time. In some examples, wireless capability detects customer arrival, which is used as a trigger to begin preparing an earlier-submitted order. Food safety may be improved by correlating food item chubs with customer profiles, to provide alert recipient information, if needed. The kiosk may be tied in with retail inventory management for automated reordering of food items and reconciling slicing operations with sales and inventory levels.

An example disclosed automated sliced food vending kiosk comprises a stock storage location for storing food item chubs; an order storage location for storing packaged food items; a slicing component operative to slice the food item chubs into sliced food items; a packaging component operative to package the sliced food items into the packaged food items; a transport component operative to transport the food item chubs from the stock storage location to the slicing component and to transport the packaged food items to the order storage location; a user interface operative to receive orders specifying the sliced food items; a dispenser component operative to dispense the packaged food items; and a control component operative to control the slicing component, the packaging component, the transport component, the user interface, and the dispenser component.

### BRIEF DESCRIPTION OF THE DRAWINGS

The disclosed examples are described in detail below with reference to the accompanying drawing figures listed below:

FIG. 1 illustrates a block diagram of an exemplary automated sliced food vending kiosk;

FIG. 2 illustrates the storage component of the automated sliced food vending kiosk of FIG. 1;

FIG. 3A illustrates the transport component of the automated sliced food vending kiosk of FIG. 1;

FIG. 3B illustrates an example gantry that may be used within the transport component of FIG. 3A;

FIGS. 4A, 4B, and 4C illustrate the slicing component of the automated sliced food vending kiosk of FIG. 1;

FIG. 5 illustrates the cleaning component of the automated sliced food vending kiosk of FIG. 1;

FIG. 6 illustrates the packaging component of the automated sliced food vending kiosk of FIG. 1;

FIG. 7 illustrates the control component of the automated sliced food vending kiosk of FIG. 1;

FIG. 8 illustrates the user interface component of the automated sliced food vending kiosk of FIG. 1;

FIG. 9 shows a flow chart illustrating exemplary operations involved in using the automated sliced food vending kiosk of FIG. 1;

FIG. 10A illustrates a front view of an example automated sliced food vending kiosk of FIG. 1;

FIG. 10B illustrates a rear view of an example of automated sliced food vending kiosk of FIG. 1; and

FIG. 11 is a block diagram of an example computing device for implementing aspects disclosed herein.

Corresponding reference characters indicate corresponding parts throughout the drawings.

### DETAILED DESCRIPTION

A more detailed understanding may be obtained from the following description, presented by way of example, in conjunction with the accompanying drawings. The entities, connections, arrangements, and the like that are depicted in, and in connection with the various figures, are presented by way of example and not by way of limitation. As such, any and all statements or other indications as to what a particular figure depicts, what a particular element or entity in a particular figure is or has, and any and all similar statements, that may in isolation and out of context be read as absolute and therefore limiting, may only properly be read as being constructively preceded by a clause such as “In at least some embodiments, . . . ” For brevity and clarity of presentation, this implied leading clause is not repeated ad nauseum.

Retail delicatessens (delis) slice blocks of meats and cheeses (called chubs) based on customer orders. Unfortunately, this common procedure has some disadvantages. It is labor intensive, requiring the deli counter to be staffed whenever a customer wishes to order sliced food items. Because the ordering occurs in person, the customer typically does not disclose identity and contact information. If shortly after the customer receives the sliced food items and leaves, there is an alert regarding a food safety issue, it may be prohibitively difficult to locate and alert the customer immediately. Additionally, because the chubs are sliced by humans, who may not record the precise amount used for each order, reconciling inventory consumption with the number of processed orders will be challenging. Deli workers may be overly generous with portions, thereby using inventory at a faster rate than reflected by the number of orders processed by the register. Conversely, deli workers may accidentally fail to provide customers with the entirety of the food purchased.

Therefore, systems and methods are disclosed that enable an automated sliced food vending kiosk to provide a sanitary, sealed environment for storing, slicing, packaging, and dispensing sliced food items, such as deli meats and cheeses. Orders may be customized through a user interface, and be scheduled for preparation to be completed coincidental with an expected approximate retrieval time. In some examples, wireless capability detects customer arrival, which is used as a trigger to begin preparing an earlier-submitted order. Food safety may be improved by correlating food item chubs with customer profiles, to provide alert recipient information, if needed. The kiosk may be tied in with retail inventory management for automated reordering of food items and reconciling slicing operations with sales and inventory levels.

An automated sliced food vending kiosk can provide customers with freshly sliced deli products, even outside the operating hours of a staffed deli, such as when the deli is closed due to low demand. The kiosk can provide samples, prepare custom orders according to customer specifications, and seal the sliced food items according to customer specifications, such as packaging meats and cheeses separately or together, and even stacked in alternating layers. Additionally, the kiosk enables improved handling of recalls when an affected chub or order can be correlated with a user account having identity and contact information. Customers may order through an app or at the kiosk itself, by selecting food items and specifying the number of slices and thicknesses or total weight. Has the ability to dispense a sample.

A tie-in with retail inventory management enables automated reordering of food items and more precisely reconciling slicing operations with sales and inventory levels. Additionally, the kiosk data provides ready insight into sales trends, which may be leveraged by an artificial intelligence (AI) capability to predict future stocking needs. Customer profile data, including order history and preferences and aversions, can be leveraged for making recommendations. Using wireless connectivity, a customer's entrance to the retail location or vicinity of the kiosk may be detected, enabling the timely preparation of the customer's order. This can reduce the risk of fulfilling an online order that is unclaimed because the customer's plans had changed and the customer fails to retrieve the order. Further, there may be additional retail space on the kiosk, such as cold storage for pre-packaged items, canceled orders that had been prepared but not collected and paid, and chub scraps.

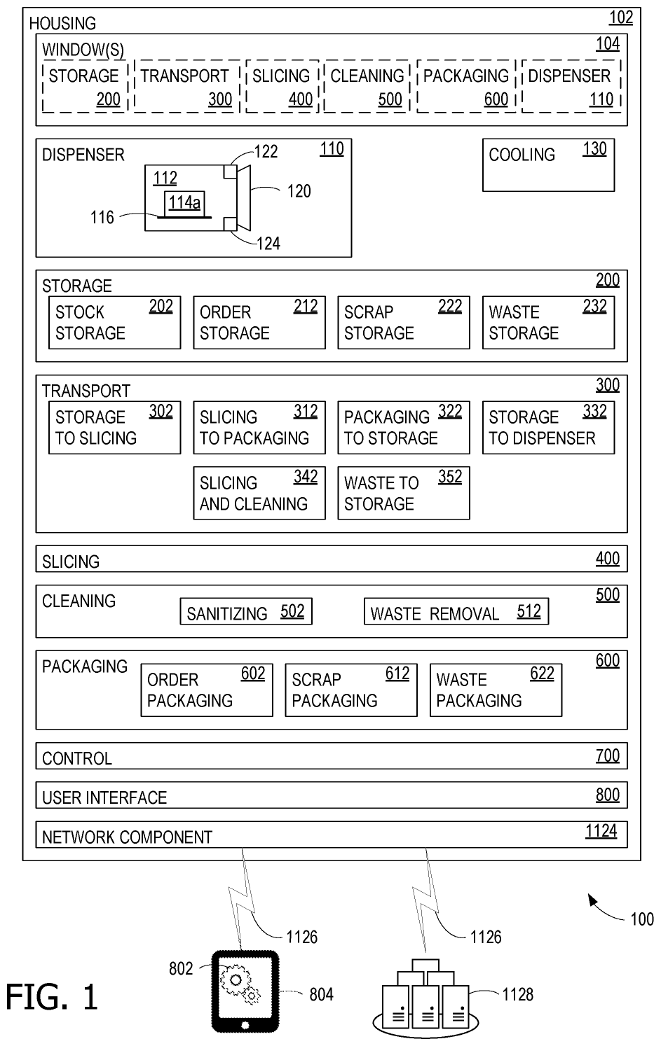

FIG. 1 illustrates a block diagram of an exemplary automated sliced food vending kiosk 100. Kiosk 100 includes a housing 102, a storage component 200, a slicing component 400 operative to slice food item chubs into sliced food items, a packaging component 600 operative to package the sliced food items into packaged food items, a transport component 300 operative to transport the food items from a stock storage location 202 to slicing component 400 and to transport the packaged food items to an order storage location 212, a user interface 800 operative to receive orders specifying the sliced food items, a dispenser component 110 operative to dispense the packaged food items; and a control component 700 operative to control slicing component 400, packaging component 600, transport component 300, user interface 800, and dispenser component 110. Storage component 200, includes stock storage location 202 for storing food item chubs and order storage location 212 for storing sliced food items.

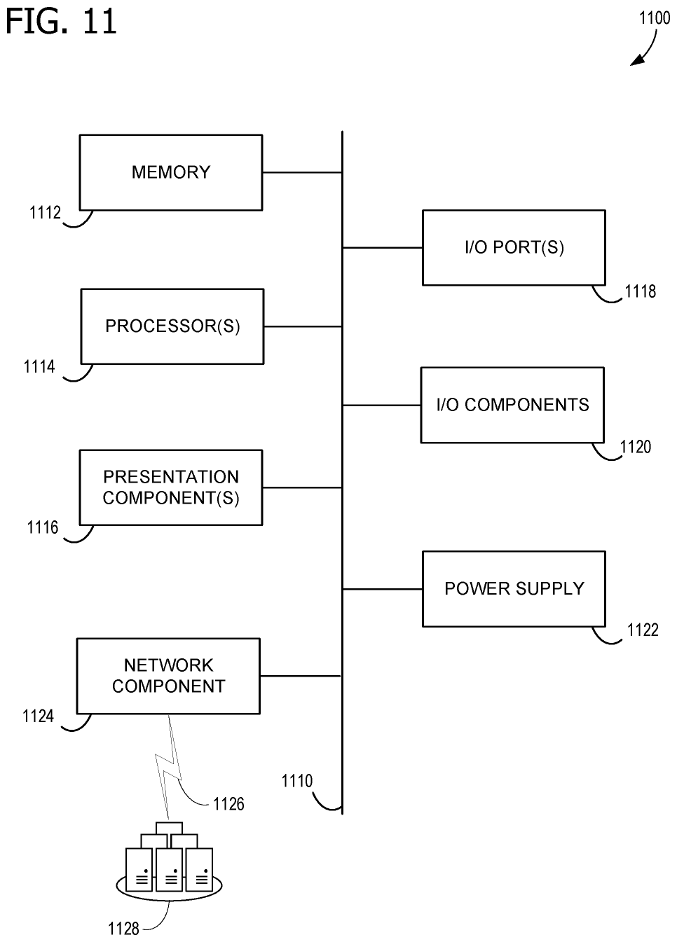

Kiosk 100 further includes a cleaning component 500. Cleaning component 500 comprises a sanitizing component 502 operative to sanitize slicing component 400. Some examples of sanitizing component 502 include an ultraviolet (UV) light. Some examples of sanitizing component 502 are operative to detect contamination of slicing component 400. Some examples of cleaning component 500 include a waste removal component 512 operative to remove food item waste produced by slicing component 400. Some examples of kiosk 100 include a waste storage location 232 for storing the food item waste. Some examples of kiosk 100 include a scrap storage location 222 for storing chub scraps, wherein packaging component 600 is further operative to package the chub scraps into packaged chub scraps, and wherein transport component 300 is further operative to transport the packaged chub scraps to scrap storage location 222. Some examples of user interface 800 include a presentation component 1116 (shown in FIG. 11).

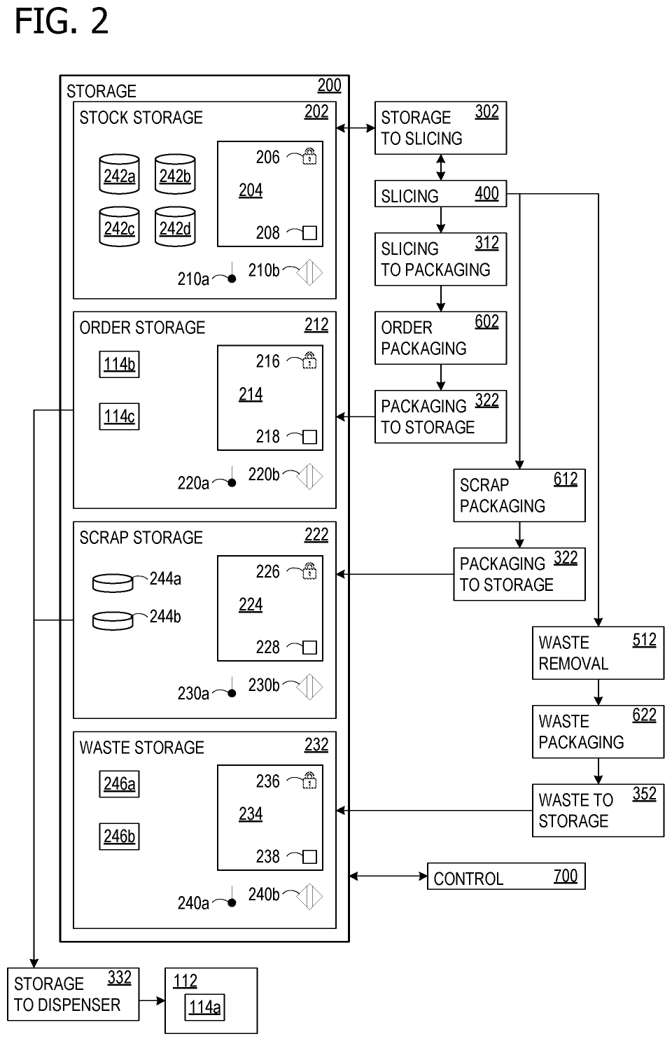

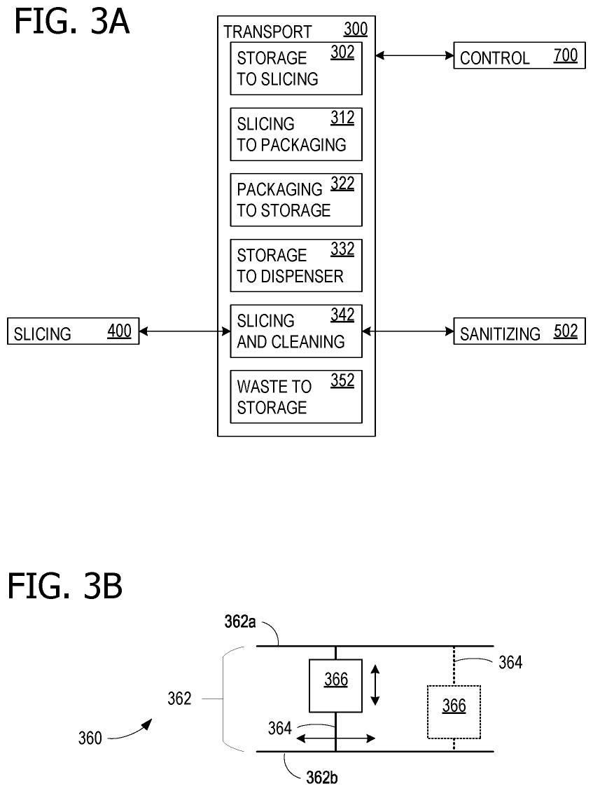

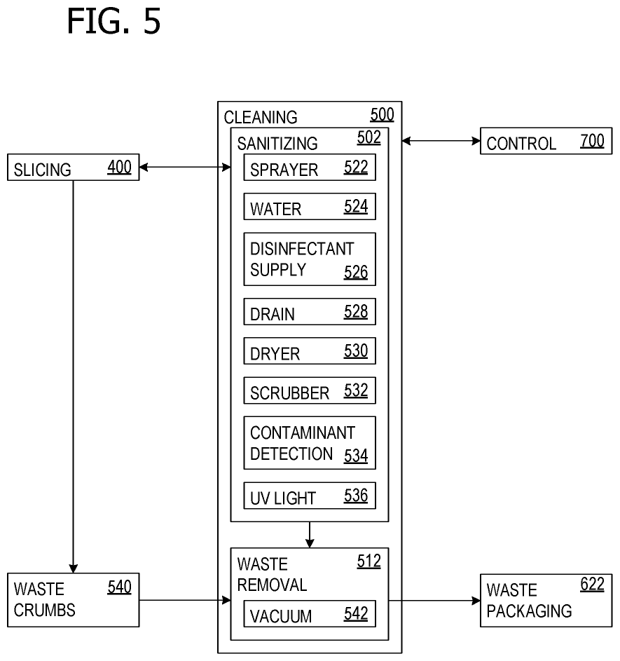

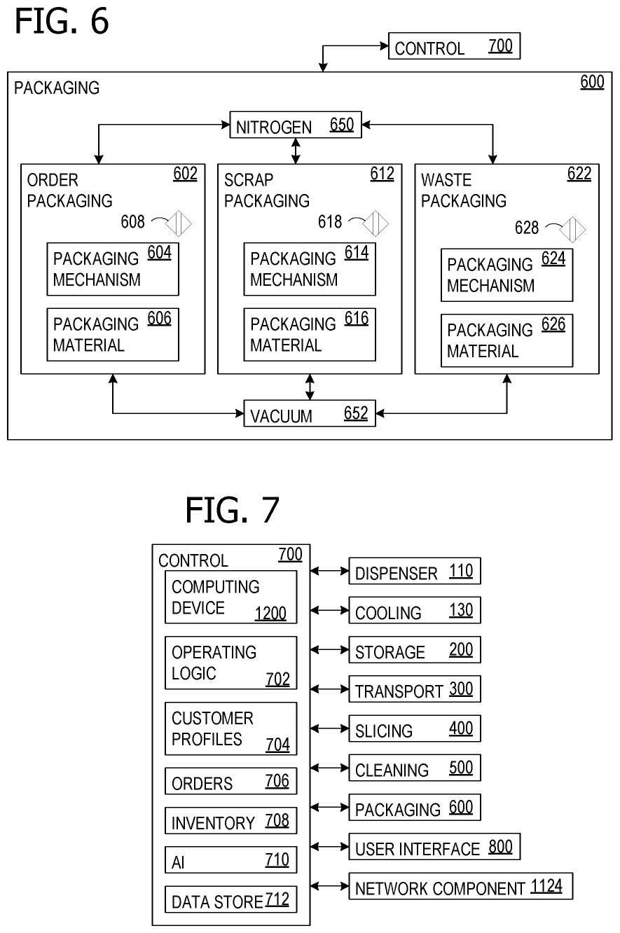

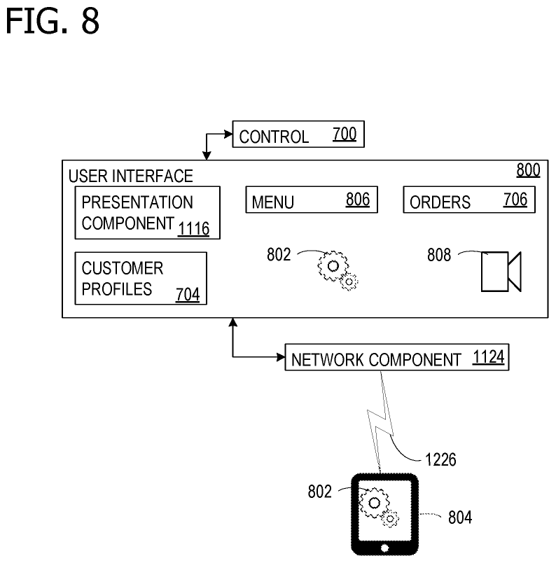

Kiosk 100 further includes a network component 1124, wherein user interface 800 comprises an application program 802 executable on a user device 804, and wherein application program 802 is operative to communicate with control component 700 through network component 1124. Storage component 200 is described in further detail in relation to FIG. 2; transport component 300 is described in further detail in relation to FIGS. 2 and 3; slicing component 400 is described in further detail in relation to FIGS. 2-4; cleaning component 500 is described in further detail in relation to FIG. 5; packaging component 600 is described in further detail in relation to FIGS. 2 and 6; control component 700 is described in further detail in relation to FIG. 7; and user interface 800 is described in further detail in relation to FIG. 8. Application program 802 and user device 804 are also described in further detail in relation to FIG. 8.

Housing 102 provides a secure, sealed environment for storage component 200, transport component 300, slicing component 400, cleaning component 500, packaging component 600, control component 700, and at least portions of user interface 800 and network component 1124. Window 104 may be a transparent wall or multiple separate viewing windows that provide customer visibility into one or more of storage component 200, transport component 300, slicing component 400, cleaning component 500, packaging component 600, and dispenser component 110. Visibility into storage component 200 permits customers to see whether certain food item chubs (e.g., meats and cheeses) are in stock, whether certain prepared orders are available, whether certain chub scraps are available, and that slicing component 400 is clean. Visibility of transport component 300 permits customers to watch the slicing, packaging (by packaging component 600), and delivery of their order to dispenser component 110, along with possibly the cleaning process of slicing component 400 by cleaning component 500.

Dispenser component 110 has a dispensing cavity 112 illustrated as holding a packaged food item 114a. A sensor 116, for example, a weight sensor, identifies that packaged food item 114a is within dispensing cavity 112, and communicates this to control component 700. Other examples of sensor 116 include a barcode scanner and other optical sensors. A dispensing door 120 (shown ajar) opens under the control of a dispensing door control 122 to permit a customer to retrieve packaged food item 114a from dispensing cavity 112. Dispensing door control 122 includes an electrically-controlled locking mechanism, controlled by control component 700, which either locks or permits opening of dispensing door 120. Dispensing door sensor 124 senses the position of dispensing door 120, for example, whether dispensing door 120 is in an open or closed position, and communicates the sensed position of dispensing door 120 to control component 700. A cooling component 130 keeps the temperature inside kiosk 100 controlled to avoid food spoilage.

As illustrated, kiosk 100 is in communication with user device 804, which is executing application program 802. Application program 802 is a portion of user interface 800, and communicates with control component 700 through network component 1124 (and also using the networking capability of user device 804), to permit a customer to place an order. Some examples of application program 802 may also use the geo-location and/or networking capability of user device 804 to signal to control component 700 that user device 804 is within the vicinity of kiosk 100, effectively a geo-fencing operation. This then can be a trigger for kiosk 100 to begin preparing the customer's internet-placed order. Had kiosk 100 prepared the order earlier, and the customer did not show up, the order may have needed to be sold at a discount, in order to prevent waste from expiration of the sliced food. Additionally, had the customer in possession of user device 804 retrieved an order for which a food safety alert was issued (e.g., a recall applicable to the food item chub sliced in preparation of the user's order), controller 700 can identify the customer and the customer's contact information from the customer profile associated with the order and alert the customer via user device 804, using network component 1124.

Also as illustrated, kiosk 100 is in communication with a remote resource 1128, via communication link 1126, using network component 1124. Remote resource 1128 may be a retail inventory management resource, additional kiosks 100, or other resources. In some examples, a single retail facility may host multiple kiosks 100, which operate cooperatively, so that if one kiosk 100 is busy, a customer order may be prepared by another co-located kiosk 100. In such examples, if a customer uses user interface on a first kiosk 100, and the customer's order is within a different kiosk 100, network component 1124 enables the first kiosk 100 to communicate with other kiosks 100 to locate the customer's order and inform the customer where to go to retrieve the order. Network component 1124, communication link 1126, and remote resource 1128 are described in further detail in relation to FIG. 11.

FIG. 2 illustrates additional detail regarding storage component 200 of kiosk 100. Storage component 200 provides a sealed environment to preserve against spoilage for storage of deli food items, which include meats, cheeses, and other types of deli food items. Stock storage location 202 holds chubs 242a-242d, which include meats and cheeses for slicing according to customer orders. Stock storage location 202 has a restricted access door 204, which is locked, to prevent unauthorized access. Restricted access door 204 can be opened by lock 206 which, in some examples, includes a mechanical lock, and in some examples is an electrically-operated mechanism controlled by control component 700. A door sensor 208 reports to control component 700 whether restricted access door 204 is opened or closed. Thus, control component 700 is operative to actuate restricted access door 204 (specifically, via lock 206) and sense a position of restricted access door 204. A temperature sensor 210a reports the temperature of stock storage location 202 to control component 700. A sensor suite 210b for stock storage location 202 is in communication with control component 700 and contains other relevant sensors, including humidity, weight of remaining food items (chubs 242a-242d), light, UV, organic chemicals (to detect signs of spoilage), quick test strips or system (to test for salmonella, bacteria, E. coli), pressure, and inventory management sensors such as barcode and/or radio frequency identification (RFID) to identify sell by dates. In some examples, sensor suite 210b includes a camera to enable a remote monitoring node to identify visible signs of spoilage.

Order storage location 212 holds packaged food items 114b and 114c, such as sliced food items prepared according to customer orders. Order storage location 212 has a restricted access door 214, which is locked, to prevent unauthorized access. Restricted access door 214 can be opened by lock 216 which, in some examples, includes a mechanical lock, and in some examples is an electrically-operated mechanism controlled by control component 700. A door sensor 218 reports to control component 700 whether restricted access door 214 is opened or closed. Thus, control component 700 is operative to actuate restricted access door 214 (specifically, via lock 216) and sense a position of restricted access door 214. A temperature sensor 220a reports the temperature of order storage location 212 to control component 700. A sensor suite 220b for order storage location 212 is in communication with control component 700 and contains other relevant sensors, such as any of the sensors indicated for sensor suite 210b.

Scrap storage location 222 holds chub scraps 244a and 244b, which are too small to be safely sliced by slicing component 400, but yet are large enough to be salable. In some examples, chub scraps 244a and 244b are packaged for sale through dispenser component 110. Scrap storage location 222 has a restricted access door 224, which is locked, to prevent unauthorized access. Restricted access door 224 can be opened by lock 226 which, in some examples, includes a mechanical lock, and in some examples is an electrically-operated mechanism controlled by control component 700. A door sensor 228 reports to control component 700 whether restricted access door 224 is opened or closed. Thus, control component 700 is operative to actuate restricted access door 224 (specifically, via lock 226) and sense a position of restricted access door 224. A temperature sensor 230a reports the temperature of scrap storage location 222 to control component 700. A sensor suite 230b for scrap storage location 222 is in communication with control component 700 and contains other relevant sensors, such as any of the sensors indicated for sensor suite 210b.

Waste storage location 232 holds waste packages 246a and 246b, holding waste from the slicing process performed by slicing component 400. Waste packages 246a and 246b are being stored until they can be removed and disposed of. Waste storage location 232 has a restricted access door 234, which is locked, to prevent unauthorized access. Restricted access door 234 can be opened by lock 236 which, in some examples, includes a mechanical lock, and in some examples is an electrically-operated mechanism controlled by control component 700. A door sensor 238 reports to control component 700 whether restricted access door 234 is opened or closed. Thus, control component 700 is operative to actuate restricted access door 234 (specifically, via lock 236) and sense a position of restricted access door 234. A temperature sensor 240a reports the temperature of waste storage location 232 to control component 700. A sensor suite 240b for waste storage location 232 is in communication with control component 700 and contains other relevant sensors, such as any of the sensors indicated for sensor suite 210b.

During operation of kiosk 100, a storage to slicing transport component 302 retrieves one of chubs 242a-242d for transport to slicing component 400. Some examples of storage to slicing transport component 302 include a robotic arm, and some examples include a conveyor, such as a belt or moving tray. After slicing, storage to slicing transport component 302 replaces the retrieved chub, if it is sufficiently unless it is too small for further slicing. In that case, the chub becomes a chub scrap and is passed to a scrap packaging component 612, where it is packaged and transported by a packaging to storage transport component 322 to scrap storage location 222.

The sliced food items are transported to an order packaging component 602 by a slicing to packaging transport component 312. In some examples, order packaging component 602 is beneath slicing component 400 and merely fall into place in order packaging component 602, although in some examples, slicing to packaging transport component 312 includes a transport mechanism such as a belt or moving tray. Packaging to storage transport component 322 passes the packaged food items to order storage location 212.

A waste removal component 512 removes waste from slicing operations and passes the waste material to a waste packaging component 622, and a waste to storage transport component 352 passes the packaged waste to waste storage location 232. More detail on waste removal component 512 is provided in relation to FIG. 5. A storage to dispenser transport component 332 retrieves packaged food items 114b and 114c and chub scraps 244a and 244b, when instructed by control component 700, and delivers them to dispenser component 110 specifically dispensing cavity 112.

FIG. 3A illustrates additional detail regarding transport component 300 of kiosk 100. Transport component 300 uses any of mechanical arms, conveyors (powered or gravity rollers), and moving trays, as are necessary for the functions described herein. Among other tasks, transport component 300 is operative to transport food items from stock storage location 202 to slicing component 400 and to transport packaged food items to order storage location 212, and is controlled by (and in communication with) control component 700. (See FIG. 2.) A slicing and cleaning transport component 342 is operative to bring slicing component 400 and sanitizing component 502 into proximity so that sanitizing component 502 can clean slicing component 400. In some examples, slicing and cleaning transport component 342 moves slicing component 400 to (possibly inside of) sanitizing component 502, whereas in some examples, slicing and cleaning transport component 342 moves sanitizing component 502 to slicing component 400. In some examples, slicing component 400 and sanitizing component 502 remain in sufficient proximity for sanitizing component 502 to clean slicing component 400, and slicing and cleaning transport component 342 is not needed. More detail on sanitizing component 502 is provided in reference to FIG. 5.

FIG. 3B illustrates an example gantry 360 that may be used within transport component 300 of kiosk 100. Gantry 360 has a track 362 that is illustrated as having a first rail 362a and a second rail 362b. A traversing rail 364 carries a trolley 366. Trolley 366 is able to move laterally (up/down, as shown) on traversing rail 364, and traversing rail 364 is able to move laterally (left/right) as shown on track 362. Different configurations and arrangements are also possible. Gantry 360 is a solution for moving mobile components within housing 102, for example, at least portions of slicing component 400 and sanitizing component 502.

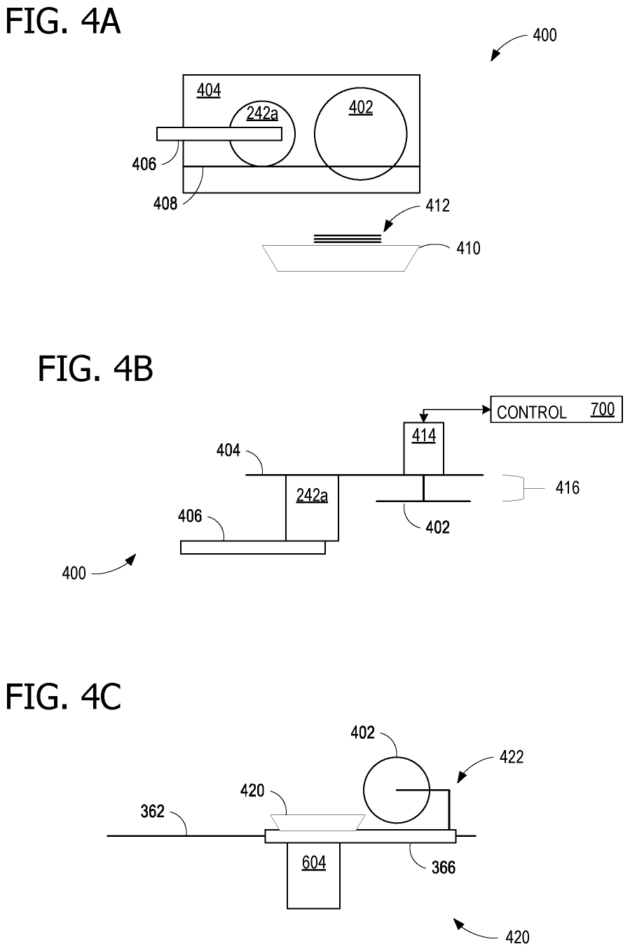

FIG. 4A illustrates a front view of slicing component 400, and FIG. 4B illustrates a top view of slicing component 400. A blade 402 slices chub 242a, which is pressed up against a table 404 by an arm 406, and resting on a shelf 408. In operation, arm 406 slides left and right (according to the perspective illustrated in FIG. 4A) moving chub 242a into the path of blade 402, then back away from blade 402 so that a subsequent slice may follow. A tray 410 waits below blade 402 to catch falling slices, which are illustrated as sliced food items 412. In some examples, another blade is operative to slice sliced food items 412 in orthogonal planes in order to produce cubed pieces of sliced food items 412. In some examples, slicing component 400 includes an additional set of blade 402, table 404, arm 406, and shelf 408 so that meats and cheeses can be sliced by different blades.

Referring now to FIG. 4B, the top view illustrates a motor 414 that is controlled by control component 700 for both operating the blade and moving blade 402 to control a cut depth 416, defined by the distance between blade 402 and table 404. In some examples, a different mechanism (such as moving table 404) is used to control cut depth 416. In some examples, slicing component 400 can vary the thickness of the slices according to the customer's order. A customer may specify a number of slices and a slice thickness, and permit the weight of sliced food items 412 to vary slightly (due to the density of the food items). Alternatively, a customer may specify a number of slices, a nominal slice thickness, an exact weight of sliced food items 412, and control component 700 will control motor 414 (or another mechanism) to slightly adjust cut depth 416 to finish the specified number of slices with the specified weight for sliced food items 412.

FIG. 4C illustrates an alternative example slicer arrangement 420 that is used with gantry 360. Slicer arrangement 420 includes blade 402 that is mounted on a moving slicer arm 422 (motor 414 and traversing rail 364 are hidden, in this view). Trolley 366 moves slicer arrangement 420 as necessary, under the control of control component 700. A tray 420 holds chubs for slicing; packaging mechanism 604 catches the slices, bags them, and seals the bags. Further detail is provided for packaging mechanism 604 in the description of FIG. 6.

FIG. 5 illustrates cleaning component 500, which includes sanitizing component 502, operative to sanitize slicing component 400, and waste removal component 512 operative to remove food item waste crumbs 540 produced by slicing component 400. Sanitizing component 502 can operate on a schedule (timed), and after every use or number of uses, and after changing chubs (e.g., after changing between meat and cheese). As illustrated, sanitizing component 502 includes a sprayer 522 that may operate similarly to a dishwasher sprayer arm. In some examples, sanitizing component 502 uses a different arrangement for spraying slicing component 400. Sprayer 522 is supplied by a water source 524 and sprays a disinfectant, such as a soap, from a disinfectant supply 526. Waste water (and some waste material) washes down a drain 528, and drying of slicing component 400 is hastened by a dryer 530, such as a heating element or air blower.

In some examples, a scrubber 532 removes stubborn waste material. Some examples of sanitizing component 502 are further operative to detect contamination of slicing component 400, using a contaminant detection component 534. Some examples of contaminant detection component 534 include test strips, and some examples include an organic chemical sensor. Contaminant detection component 534 is operative to detect one or more of salmonella, Listeria monocytogenes (listeria), E. coli, other bacteria. In some examples, contaminant detection component 534 is a camera, monitored by remote resource 1128 (see FIG. 1). The illustrated example of sanitizing component 502 further comprises a UV light 536 that can neutralize certain harmful organic material. As illustrated, waste removal component 512 includes a vacuum 542 to vacuum up waste crumbs 540 and deposit them in waste packaging component 622, although other mechanisms (such as wipers) may also be used to remove waste crumbs 540.

FIG. 6 illustrates additional detail for packaging component 600, which is coupled to and controlled by control component 700. Order packaging component 602 includes a packaging mechanism 604 that is operative to package sliced food items 412 into packaged food items, such as one of packaged food items 114a-114c. Order packaging component 602 also includes packaging material 606, such as plastic bags, and a sensor suite 608. Sensor suite 608 is in communication with control component 700 and contains relevant sensors, such as any of the sensors indicated for sensor suite 210b (of FIG. 2). An example of a sensor in sensor suite 608 is a bag full sensor that triggers control component 700 to operate transport component 300 to remove a full bag.

Scrap packaging component 612 includes a packaging mechanism 614 that is operative to package chub scraps 244a and 244b. Scrap packaging component 612 also includes packaging material 616, such as plastic bags, and a sensor suite 618. Sensor suite 618 is in communication with control component 700 and contains relevant sensors, such as any of the sensors indicated for sensor suite 210b. Waste packaging component 622 includes a packaging mechanism 624 that is operative to package waste crumbs 540 received from waste removal component 512. Waste packaging component 622 also includes packaging material 626, such as plastic bags, and a sensor suite 628. Sensor suite 628 is in communication with control component 700 and contains relevant sensors, such as any of the sensors indicated for sensor suite 210b.

A nitrogen charging component 650 charges certain portions of packaging component 600 to insert nitrogen into the bags prior to vacuum sealing the bags with vacuum packing component 652. Nitrogen can help preserve the packaged food items and waste material against spoliation. In some examples, another inert gas, rather than or in addition to nitrogen, may be used.

FIG. 7 illustrates control component 700 in greater detail. Control component is in communication with and operative to control dispenser component 110, cooling component 130, storage component 200, transport component 300, slicing component 400, cleaning component 500, packaging component 600, user interface 800, and network component 1124. Some examples of control component include a computing device 1100. More detail regarding computing device 1100 is provided with reference to FIG. 11.

Control component 700 includes an operating logic 702 that controls the other components as described herein. For example, operating logic 702 enables improved handling of recalls and food safety alerts by using contact information within customer profiles 704 to locate customers who have purchased affected food items. Additionally, because food safety alerts may come in through network component 1124 or be generated locally when detecting contamination, kiosk 100 can immediately quarantine affected food chubs without risk of human error permitting a mistaken serving. That is operating logic 702 correlates affected food item chubs with customer profiles 704, to provide alert recipient information, if needed.

In some examples, operating logic 702 can optimize food item preparation time by scheduling the slicing and packaging processes to be completed coincidental with an expected approximate retrieval time. If a customer is waiting at kiosk 100, then preparation may begin immediately. However, if the order is placed online, and the customer is not in the vicinity of the kiosk (e.g., within the retail location facility), there is a chance that the customer's plans may change and the customer will not arrive to pick up the order prior to food safety expiration of the order. If the order had been prepared, and he customer did not arrive in sufficient time, then operating logic 702 will mark the price down and offer the unclaimed order at a discount. In some examples, an advertisement of the mark-down will be sent over network component 1124.

However, to reduce the likelihood of that occurrence, operating logic 702 can wait until the customer's presence is detected within the vicinity of kiosk 100, for example by using a wireless sensor to detect user device 804. In some examples, application program 802 assists with the detection of user device 804 by attempting to contact kiosk 100 over network component 1124. At that time, operating logic 702 begins the preparation of the order. Alternatively, if the order is part of an online grocery pickup (OGP) order, then the walk time of the shopper (e.g., an employee of the retail facility in which kiosk 100 is located) can be predicted, and the order prepared at approximately the time that the shopper arrives at kiosk 100. This can help maximize freshness of the food items when the customer arrives to retrieve the OGP order. The orders awaiting retrieval and preparation (processing) are stored in an order data set 706.

The inventory within kiosk 100 is cataloged in inventory data 708, and can be tied in with the larger inventory data set of the retail facility in which kiosk 100 is located. Operating logic 702 can track the usage rate of various chubs within stock storage location 202, the remaining amount, and the upcoming expiration date. If it is likely that there may be product expiration prior to depletion, then operating logic 702 will mark down the price of the orders using the affected chubs, and advertise the discount using network component 1124. Additionally, operating logic 702 can automatically request restocking when the onboard inventory of chubs runs low. Further, operating logic 702 can reconcile slicing operations with sales and inventory levels. Because the sliced food items were weighed, operating logic 702 can reconcile slicing operations with sales and inventory levels more accurately than a hand slicing operation s able. An AI component 710 can leverage the history of inventory data 708 to predict future stocking needs, as well optimize cleaning intervals. A data store 712 stores other data related to the operations of kiosk 100.

FIG. 8 illustrates user interface 800 of kiosk 100 in greater detail. User interface 800 comprises presentation component 1116, application program 802, a menu 806, order data set 706, customer profiles 704, and a barcode scanner/camera 808. The arrangement illustrated for user interface 800 overlaps with other components, for example, application program 802 resides on user device 804, and order data set 706, and customer profiles 704 reside within control component 700. With user interface 800, a customer may place an order online, at kiosk 100 using presentation component 1116 (which, in some examples includes a touchscreen), or with application program 802 on user device 804. Barcode scanner/camera 808 reads barcodes displayed on user device 804 when the customer is retrieving an order, and/or barcodes on packaged food items 114a-114c and chub scraps 244a-244b to confirm retrieval from dispensing cavity 112. In some examples, presentation component 1116 is also used for displaying messages relevant to operating kiosk 100, for example, “Cleaning the cutting wheel now” and “Confirmed zero bacterial presence.” Status of orders, retrieved from order data set 706 are also displayed on presentation component 1116.

Menu 806 provides recommendations and allows for customization of orders. For example, menu 806 pairs complementary products (meats and cheese), as well as interface with customers for feedback. Customer profiles 704 stores order histories and favorites for known customers, which can be used for suggestions in conjunction with menu 806. Input into user interface 800 for an order may include, for example, ten instances of alternating two slices of ham with a single slice of cheese, in order to produce material for ten sandwiches. Alternatively, an input into user interface 800 may be to repeat a previous order. Menu 806 further provides the option of selecting the food items by the name of a sandwich, if the customer does not know the exact ingredients. Additionally, menu 806 suggests additional ingredients needed to finish certain types of sandwiches, as well as where to find them in the retail facility, and the amount needed. For example, menu 806 may suggest sandwich bread from the bakery department, red onions from produce, and sliced dill pickles on a particular aisle.

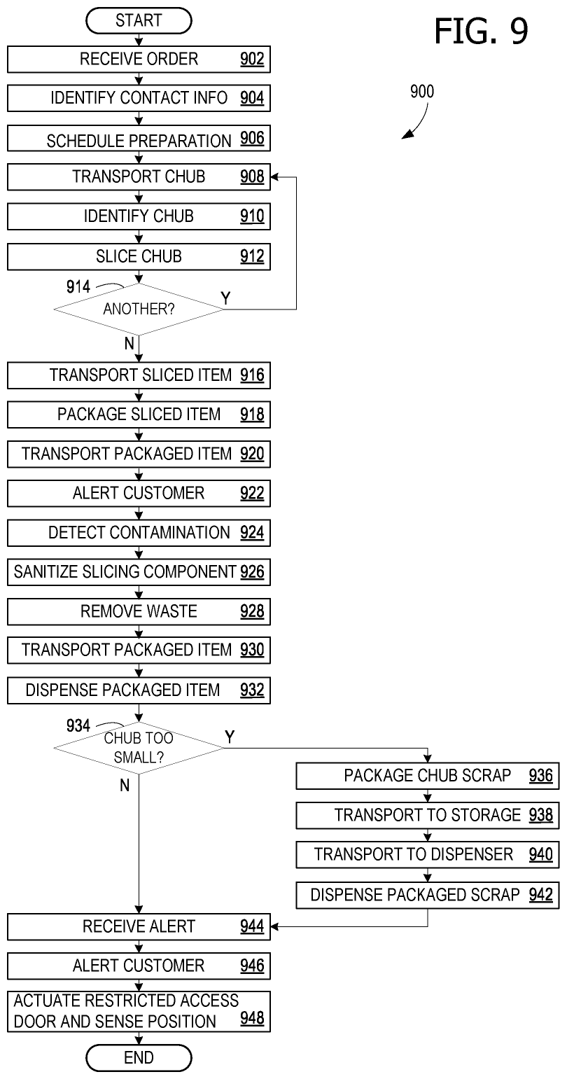

FIG. 9 shows a flow chart 900 illustrating exemplary operations involved in using kiosk 100. With flow chart 900 customer orders may be customized through a user interface, and be scheduled for preparation to be completed coincidental with an expected approximate retrieval time. In some examples, wireless capability detects customer arrival, which is used as a trigger to begin preparing an earlier-submitted order. Food safety may be improved by correlating food item chubs with customer profiles, to provide alert recipient information, if needed. The kiosk may be tied in with retail inventory management for automated reordering of food items and reconciling slicing operations with sales and inventory levels.

Operation 902 includes receiving an order specifying a sliced food item. Some examples of operation 902 include receiving the order through an application program that is operative to communicate with a control component of the kiosk. Some examples of operation 902 include receiving the order through a presentation component on the kiosk. Operation 904 includes identifying contact information for a customer placing the received order. The contact information may be located in a customer profile and can assist with notifying the customer in the event of a food safety alert. Operation 906 includes scheduling preparation of a packaged food item based at least on an expected approximate retrieval time of the packaged food item. This may be based on an OGP operation, or the customer arriving in the vicinity of the kiosk. If the customer is at the kiosk, then preparation of the order may begin immediately.

Operation 908 includes transporting a food item chub from a stock storage location to a slicing component, and operation 912 includes, with the slicing component, slicing the food item chub into the sliced food item. Decision operation 914 determines whether another chub is needed for the order, and if so, flow chart 900 returns to operation 908. In some examples, a single chub may be used multiple times, if the order has specified alternating slices from different chubs (e.g., alternating a single meet with a single cheese, in layers). Operation 916 includes transporting the sliced food item to a packaging component. In some examples, this may involve the sliced food items merely falling onto the packaging component, although in other examples, a conveyor is used. Operation 918 includes packaging the sliced food item into a packaged food item, and operation 920 includes transporting the packaged food item to an order storage location. Operation 922 includes alerting the customer to retrieve the packaged food item, perhaps using the application program that placed the order.

Operation 924 includes detecting contamination of the slicing component, to ensure food safety, and operation 926 includes automatically sanitizing the slicing component. Operation 926 may be performed on a set time schedule, after each order (or a set number of orders), when changing a chub, or upon operation 924 detecting contamination. In some examples, operation 926 also includes transporting the sanitizing component to the slicing component, or transporting the slicing component to the sanitizing component. Operation 928 includes automatically removing food item waste produced by the slicing component. Some examples of operation 928 include packaging the waste, transporting the waste to a waste storage location, and storing the waste until disposal.

When the customer arrives to retrieve the order, operation 930 includes transporting the packaged food item from the order storage location to a dispenser component. Operation 932 then includes dispensing the packaged food item from the dispenser component. In order to improve efficiency, when a chub is too small to use in the slicing component, yet large enough that some customers may wish to purchase it, it can be packaged and sold. Therefore, decision operation 934 determines whether a chub is too small for further slicing. If it is, then operation 936 includes packaging the chub scrap into packaged chub scraps, and operation 938 includes transporting the packaged chub scrap to a scrap storage location. When a customer wishes to purchase the chub scrap, operation 940 includes transporting the packaged chub scrap from the scrap storage location to the dispenser component, and operation 942 includes dispensing the packaged chub scrap from the dispenser component. In the unfortunate event that a food safety alert is received in operation 944, operation 946 includes alerting the customer using the contact information obtained in operation 904. Operation 948 includes actuating a restricted access door and sensing its position (open or closed) to permit cleaning, resupply, or maintenance (e.g., restricted access door 204 controlled by lock 206, or any of restricted access doors 214, 224, and 234).

In some examples, the operations illustrated in flow chart 900 may be implemented as software instructions encoded on a computer readable medium, in hardware programmed or designed to perform the operations, or both. For example, aspects of the disclosure may be implemented as a system on a chip or other circuitry including a plurality of interconnected, electrically conductive elements. While the aspects of the disclosure have been described in terms of various examples with their associated operations, a person skilled in the art would appreciate that a combination of operations from any number of different examples or some reordering is also within scope of the aspects of the disclosure.

### Exemplary Operating Environment

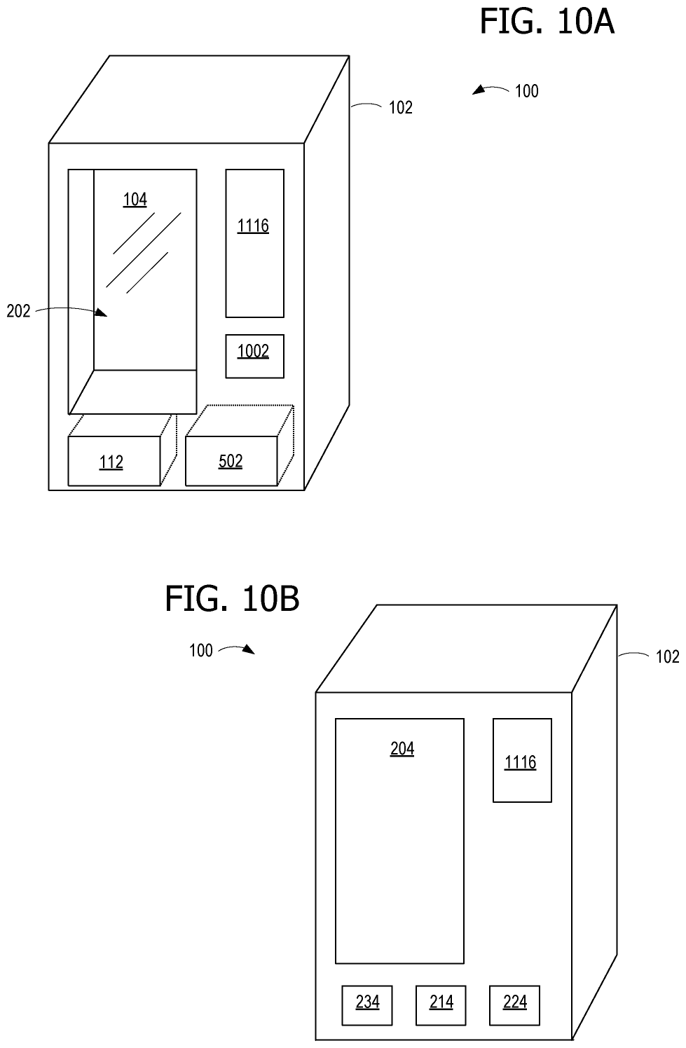

FIG. 10A illustrates a front view of an example automated sliced food vending kiosk 100, and FIG. 10B illustrates a rear view of an example automated sliced food vending kiosk 100. In FIG. 10A, the front of housing 102 holds window 104, which permits customers to see inside of stock storage location 202. Customers can input orders and select products via adjacent presentation component 1116, and pay with a payment portal 1002 that accepts cash, credit cards, mobile phone payments, and other forms of payments. Dispensing cavity 112 is illustrated beneath stock storage location 202, but may be located elsewhere in other examples. Sanitizing component 502 is illustrated nearby dispensing cavity 112, although in some examples, sanitizing component 502 is mobile within housing 102, for example by using gantry 360 (of FIG. 3).

In FIG. 10B, the rear of housing 102 shows restricted access doors 204, 234, 214, and 224, arranged as shown, although other arrangements may be used, depending on the configuration of components within housing 102. Another presentation component 1116 is provided to enable maintenance, cleaning, and restocking of n interface 1004, for example a touchscreen interface. Presentation component 1116 on the rear of housing 102 is used, for example, in instruct control component 700 to actuate one or more of restricted access doors 204-234.

FIG. 11 is a block diagram of an example computing device 1100 for implementing aspects disclosed herein. That is, computing device 1100 comprises a computer storage device having computer-executable instructions stored thereon, which, on execution by a computer, cause the computer to perform operations described herein. Computing device 1100 is one example of a suitable computing environment and is not intended to suggest any limitation as to the scope of use or functionality of the invention. Neither should the computing device 1100 be interpreted as having any dependency or requirement relating to any one or combination of components/modules illustrated.

The examples and embodiments disclosed herein may be described in the general context of computer code or machine-useable instructions, including computer-executable instructions such as program components, being executed by a computer or other machine, such as a personal data assistant or other handheld device. Generally, program components including routines, programs, objects, components, data structures, and the like, refer to code that performs particular tasks, or implement particular abstract data types. The disclosed examples may be practiced in a variety of system configurations, including personal computers, laptops, smart phones, mobile tablets, hand-held devices, consumer electronics, specialty computing devices, etc. The disclosed examples may also be practiced in distributed computing environments, where tasks are performed by remote-processing devices that are linked through a communications network.

Computing device 1100 includes a bus 1110 that directly or indirectly couples the following devices: memory 1112, one or more processors 1114, one or more presentation components 1116, input/output (I/O) ports 1118, I/O components 1120, a power supply 1122, and a network component 1124. Computing device 1100 should not be interpreted as having any dependency or requirement related to any single component or combination of components illustrated therein. While computing device 1100 is depicted as a seemingly single device, multiple computing devices 1100 may work together and share the depicted device resources. That is, one or more computer storage devices having computer-executable instructions stored thereon may perform operations disclosed herein. For example, memory 1112 may be distributed across multiple devices, processor(s) 1114 may provide housed on different devices, and so on.

Bus 1110 represents what may be one or more busses (such as an address bus, data bus, or a combination thereof). Although the various blocks of FIG. 11 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. Such is the nature of the art, and the diagram of FIG. 11 is merely illustrative of an exemplary computing device that can be used in connection with one or more embodiments. Distinction is not made between such categories as “workstation,” “server,” “laptop,” “hand-held device,” etc., as all are contemplated within the scope of FIG. 11 and the references herein to a “computing device.” Memory 1112 may include any of the computer-readable media discussed herein. Memory 1112 may be used to store and access instructions configured to carry out the various operations disclosed herein. In some examples, memory 1112 includes computer storage media in the form of volatile and/or nonvolatile memory, removable or non-removable memory, data disks in virtual environments, or a combination thereof.

Processor(s) 1114 may include any quantity of processing units that read data from various entities, such as memory 1112 or I/O components 1120. Specifically, processor(s) 1114 are programmed to execute computer-executable instructions for implementing aspects of the disclosure. The instructions may be performed by the processor, by multiple processors within the computing device 1100, or by a processor external to the client computing device 1100. In some examples, the processor(s) 1114 are programmed to execute instructions such as those illustrated in the flow charts discussed below and depicted in the accompanying drawings. Moreover, in some examples, the processor(s) 1114 represent an implementation of analog techniques to perform the operations described herein. For example, the operations may be performed by an analog client computing device 1100 and/or a digital client computing device 1100.

Presentation component(s) 1116 present data indications to a user or other device. Exemplary presentation components 1116 include a display device, speaker, printing component, vibrating component, etc. One skilled in the art will understand and appreciate that computer data may be presented in a number of ways, such as visually in a graphical user interface (GUI), audibly through speakers, wirelessly between computing devices 1100, across a wired connection, or in other ways. Ports 1118 allow computing device 1100 to be logically coupled to other devices including I/O components 1120, some of which may be built in. Example I/O components 1120 include, for example but without limitation, a microphone, keyboard, mouse, joystick, game pad, satellite dish, scanner, printer, wireless device, etc.

In some examples, the network component 1124 includes a network interface card and/or computer-executable instructions (e.g., a driver) for operating the network interface card. Communication between the computing device 1100 and other devices may occur using any protocol or mechanism over any wired or wireless connection. In some examples, the network component 1124 is operative to communicate data over public, private, or hybrid (public and private) using a transfer protocol, between devices wirelessly using short range communication technologies (e.g., near-field communication (NFC), Bluetooth® branded communications, or the like), or a combination thereof. Network component 1124 communicates over communication link 1126 to a remote resource 1128. Various different examples of communication link 1126 include a wired connection, wireless connection, and/or a dedicated link, and in some examples, at least a portion is routed through the internet. Various different examples of remote resource 1128 include computational services in support of the functionality described herein, and may include cloud computing and/or storage resources. Examples of remote resource 1128 may themselves be assembled using examples of computing node 1100.

Although described in connection with an example computing device 1100, examples of the disclosure are capable of implementation with numerous other general-purpose or special-purpose computing system environments, configurations, or devices. Examples of well-known computing systems, environments, and/or configurations that may be suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, virtual reality (VR) devices, holographic device, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more or less functionality than illustrated and described herein. In examples involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals per se. Exemplary computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that can be used to store information for access by a computing device. In contrast, communication media typically embody computer readable instructions, data structures, program modules, or the like in a modulated data signal such as a carrier wave or other transport mechanism and include any information delivery media.

### Exemplary Operating Methods and Systems

An exemplary automated sliced food vending kiosk comprises a stock storage location for storing food item chubs; an order storage location for storing packaged food items; a slicing component operative to slice the food item chubs into sliced food items; a packaging component operative to package the sliced food items into the packaged food items; a transport component operative to transport the food item chubs from the stock storage location to the slicing component and to transport the packaged food items to the order storage location; a user interface operative to receive orders specifying the sliced food items; a dispenser component operative to dispense the packaged food items; and a control component operative to control the slicing component, the packaging component, the transport component, the user interface, and the dispenser component.

An exemplary method for operating an automated sliced food vending kiosk, implemented on at least one processor, comprises: receiving an order specifying a sliced food item; transporting a food item chub from a stock storage location to a slicing component; with the slicing component, slicing the food item chub into the sliced food item; packaging the sliced food item into a packaged food item; transporting the packaged food item to an order storage location; transporting the packaged food item from the order storage location to a dispenser component; and dispensing the packaged food item from the dispenser component.

One or more exemplary computer storage devices having a first computer-executable instructions stored thereon for operating an automated sliced food vending kiosk, which, on execution by a computer, cause the computer to perform operations which comprise: receiving an order specifying a sliced food item through an application program that is operative to communicate with a control component of the kiosk; identifying contact information for a customer placing the received order; scheduling preparation of a packaged food item based at least on an expected approximate retrieval time of the packaged food item; transporting a food item chub from a stock storage location to a slicing component; with the slicing component, slicing the food item chub into the sliced food item; packaging the sliced food item into the packaged food item; transporting the packaged food item to an order storage location; alerting customer to retrieve the packaged food item; detecting contamination of the slicing component; automatically sanitizing the slicing component; automatically removing food item waste produced by the slicing component; transporting the packaged food item from the order storage location to a dispenser component; dispensing the packaged food item from the dispenser component; packaging a chub scrap into a packaged chub scrap; transporting the packaged chub scrap to a scrap storage location; transporting the packaged chub scrap from the scrap storage location to the dispenser component; dispensing the packaged chub scrap from the dispenser component; receiving a food safety alert; alerting the customer using the contact information; and actuating a restricted access door and sensing its position.

Alternatively, or in addition to the other examples described herein, examples include any combination of the following:
a cleaning component, wherein the cleaning component comprises a sanitizing component operative to sanitize the slicing component;
the sanitizing component comprises a UV light;
the sanitizing component is further operative to detect contamination of the slicing component;
the cleaning component further comprises a waste removal component operative to remove food item waste produced by the slicing component;
a waste storage location for storing the food item waste;
a scrap storage location for storing chub scraps, wherein the packaging component is further operative to package the chub scraps into packaged chub scraps, and wherein the transport component is further operative to transport the packaged chub scraps to the scrap storage location;
the user interface comprises a presentation component;
the user interface comprises a presentation component;
a network component, wherein the user interface comprises an application program executable on a user device, and wherein the application program is operative to communicate with the control component through the network component;
receiving the order through an application program that is operative to communicate with a control component of the kiosk;
identifying contact information for a customer placing the received order;
receiving a food safety alert; and alerting the customer using the contact information;
alerting customer to retrieve the packaged food item;
scheduling preparation of the packaged food item based at least on an expected approximate retrieval time of the packaged food item;
detecting contamination of the slicing component;
automatically sanitizing the slicing component;
automatically removing food item waste produced by the slicing component;
packaging a chub scrap into a packaged chub scrap; transporting the packaged chub scrap to a scrap storage location; transporting the packaged chub scrap from the scrap storage location to the dispenser component; and dispensing the packaged chub scrap from the dispenser component; and
actuating a restricted access door and sensing its position.

The order of execution or performance of the operations in examples of the disclosure illustrated and described herein may not be essential, and thus may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense. While the disclosure is susceptible to various modifications and alternative constructions, certain illustrated examples thereof are shown in the drawings and have been described above in detail. It should be understood, however, that there is no intention to limit the disclosure to the specific forms disclosed, but on the contrary, the intention is to cover all modifications, alternative constructions, and equivalents falling within the spirit and scope of the disclosure.

What is claimed is:

1. An automated sliced food vending kiosk comprising:
a stock storage location for storing food item chubs;
an order storage location for storing packaged food items;
a slicing component operative to slice the food item chubs into sliced food items;
a packaging component operative to package the sliced food items into the packaged food items;
a transport component operative to transport the food item chubs from the stock storage location to the slicing component and to transport the packaged food items to the order storage location;
a user interface operative to receive orders specifying the sliced food items;
a dispenser component operative to dispense the packaged food items; and
a control component operative to control the slicing component, the packaging component, the transport component, the user interface, and the dispenser component.

2. The kiosk of claim 1 further comprising:
a cleaning component, wherein the cleaning component comprises a sanitizing component operative to sanitize the slicing component.

3. The kiosk of claim 2 wherein the sanitizing component comprises an ultraviolet (UV) light.

4. The kiosk of claim 2 wherein the sanitizing component is further operative to detect contamination of the slicing component.

5. The kiosk of claim 2 wherein the cleaning component further comprises:
a waste removal component operative to remove food item waste produced by the slicing component; and
a waste storage location for storing the food item waste.

6. The kiosk of claim 1 wherein the control component is further operative to actuate a restricted access door and sense a position of the restricted access door.

7. The kiosk of claim 1 further comprising:
a scrap storage location for storing chub scraps,
wherein the packaging component is further operative to package the chub scraps into packaged chub scraps, and
wherein the transport component is further operative to transport the packaged chub scraps to the scrap storage location.

8. The kiosk of claim 1 wherein the user interface comprises a presentation component.

9. The kiosk of claim 1 further comprising:
a network component,
wherein the user interface comprises an application program executable on a user device, and
wherein the application program is operative to communicate with the control component through the network component.

10. A method for operating an automated sliced food vending kiosk, the method comprising:
receiving an order specifying a sliced food item;
transporting a food item chub from a stock storage location to a slicing component;
with the slicing component, slicing the food item chub into the sliced food item;
packaging the sliced food item into a packaged food item;
transporting the packaged food item to an order storage location;
transporting the packaged food item from the order storage location to a dispenser component; and
dispensing the packaged food item from the dispenser component.

11. The method of claim 10 further comprising:
receiving the order through an application program that is operative to communicate with a control component of the kiosk.

12. The method of claim 10 further comprising:
identifying contact information for a customer placing the received order.

13. The method of claim 12 further comprising:
receiving a food safety alert; and
alerting the customer using the contact information.

14. The method of claim 10 further comprising:
alerting customer to retrieve the packaged food item.

15. The method of claim 10 further comprising:
scheduling preparation of the packaged food item based at least on an expected approximate retrieval time of the packaged food item.

16. The method of claim 10 further comprising:
detecting contamination of the slicing component.

17. The method of claim 10 further comprising:
automatically sanitizing the slicing component.

18. The method of claim 10 further comprising:
automatically removing food item waste produced by the slicing component.

19. The method of claim 10 further comprising:
packaging a chub scrap into a packaged chub scrap;
transporting the packaged chub scrap to a scrap storage location;
transporting the packaged chub scrap from the scrap storage location to the dispenser component; and
dispensing the packaged chub scrap from the dispenser component.

20. One or more computer storage devices having computer-executable instructions stored thereon for operating an automated sliced food vending kiosk, which, on execution by a computer, cause the computer to perform operations comprising:
receiving an order specifying a sliced food item through an application program that is operative to communicate with a control component of the kiosk;
identifying contact information for a customer placing the received order;
scheduling preparation of a packaged food item based at least on an expected approximate retrieval time of the packaged food item;
transporting a food item chub from a stock storage location to a slicing component;
with the slicing component, slicing the food item chub into the sliced food item;
packaging the sliced food item into the packaged food item;
transporting the packaged food item to an order storage location;
alerting customer to retrieve the packaged food item;
detecting contamination of the slicing component;
automatically sanitizing the slicing component;
automatically removing food item waste produced by the slicing component;
transporting the packaged food item from the order storage location to a dispenser component;
dispensing the packaged food item from the dispenser component;
packaging a chub scrap into a packaged chub scrap;
transporting the packaged chub scrap to a scrap storage location;
transporting the packaged chub scrap from the scrap storage location to the dispenser component;
dispensing the packaged chub scrap from the dispenser component;
receiving a food safety alert;
alerting the customer using the contact information; and
actuating a restricted access door and sensing its position.
